(19) United States
(12) Patent Application Publication
(10) Pub. No.: US 2025/0032544 A1
(43) Pub. Date: Jan. 30, 2025

(54) SIRPALPHA SWITCH RECEPTORS

(71) Applicant: THE REGENTS OF THE UNIVERSITY OF CALIFORNIA, Oakland, CA (US)

(72) Inventor: Tobias Deuse, Burlingame, CA (US)

(73) Assignee: THE REGENTS OF THE UNIVERSITY OF CALIFORNIA, Oakland, CA (US)

(21) Appl. No.: 18/716,906

(22) PCT Filed: Dec. 7, 2022
(86) PCT No.: PCT/US22/52062

§ 371 Date: Jun. 5, 2024

Related U.S. Application Data
(60) Provisional application No. 63/288,878, filed on Dec. 13, 2021.

(51) Int. Cl. A61K 35/17; A61K 39/00; A61P 35/02; C07K 14/705

(52) U.S. Cl. CPC A61K 35/17; A61K 39/4611; A61K 39/4613; A61K 39/4631; A61K 39/464429; A61P 35/02; C07K 14/70596; A61K 2239/15; A61K 2239/21; A61K 2239/22; A61K 2239/48

(57) ABSTRACT

The invention provides recombinant CD47 engager receptor proteins comprising an intracellular domain that enhances an immune response in a cell when the CD47 engager receptor protein binds to CD47 on a target cell. The invention also provides cells comprising the CD47 engager receptor proteins. The CD47 engager receptor protein may be a switched Signal Regulatory Protein Alpha (SIRPα) receptor (SIRPα switch receptors) and cells that comprise them (SIRPα switch cells). SIRPα switch receptors recognize CD47 on target tumor cells and transmit an intracellular signal within the SIRPα switch cells that upregulates natural killer cell (NK), T cell, and macrophage activity. The result is increased tumor cell killing.

### I. CROSS REFERENCE TO RELATED APPLICATIONS

This application claims priority under 35 U.S.C. § 119(e) to U.S. Provisional Application No. 63/288,878, filed Dec. 13, 2021, and is incorporated herein by reference in its entirety.

### II. FIELD OF THE INVENTION

The invention provides recombinant CD47 engager receptor proteins comprising an intracellular domain that enhances an immune response in a cell when the CD47 engager receptor protein binds to CD47 on a target cell. The invention also provides cells comprising the CD47 engager receptor proteins. The CD47 engager receptor protein may be a switched Signal Regulatory Protein Alpha (SIRPα) receptor (SIRPα switch receptors) and cells that comprise them (SIRPα switch cells). SIRPα switch receptors recognize CD47 on target tumor cells and transmit an intracellular signal within the SIRPα switch cells that upregulates natural killer cell (NK), T cell, and macrophage activity. The result is increased tumor cell killing.

### III. BACKGROUND OF THE INVENTION

Natural killer cells, or NK cells, are cytotoxic lymphocytes critical to the innate immune system. The role NK cells play is analogous to that of cytotoxic T cells in the vertebrate adaptive immune response. NK cells provide rapid responses to virus-infected and cancerous cells. Typically, NK cells become activated by target cells downregulating major histocompatibility complex (MHC) as this is one major inhibitory NK cell signal. NK cell activation triggers cytokine and granule release resulting in lysis or apoptosis. NK cells are unique, because they can recognize stressed cells as they upregulate other stimulatory NK cell signals and do not require prior exposure to certain cell epitopes. This makes them very fast responders. They can also quickly respond to antibody-laden cells because binding to antibody Fc is a strong stimulatory NK cell signal. NK cells do not require major activation to kill cells that are missing “self” markers of MHC class 1 other than some cytokine exposure like IL-2 or IL-15. This role is especially important because harmful cells that have downregulated or missing MHC I markers cannot be detected and destroyed by other immune cells such as T lymphocyte cells.

NK cells are large granular lymphocytes that are differentiated from the common lymphoid progenitor-generating B and T lymphocytes. They differentiate and mature in the bone marrow, lymph nodes, spleen, tonsils, and thymus, where they then enter into the circulation.

SIRPα is a member of the signal-regulatory-protein (SIRP) family and also belongs to the immunoglobulin superfamily. SIRP family members are receptor-type transmembrane glycoproteins known to be involved in the negative regulation of receptor tyrosine kinase-coupled signaling processes. SIRPα can be phosphorylated by tyrosine kinases. The phosphotyrosine residues recruit SH2 domain-containing tyrosine phosphatases (PTP) and serve as their substrates. SIRPα participates in signal transduction mediated by various growth factor receptors.

CD47 is a ligand for SIRPα. CD47 is a “marker-of-self” protein that can be overexpressed broadly across tumor types. It is emerging as a novel potent macrophage and NK cell immune checkpoint for cancer immunotherapy. CD47 in tumor cells sends a “don't-eat-me” signal that inhibits macrophage phagocytosis. This presents opportunities and challenges for CD47 inhibitors both as a monotherapy and in combination treatments for hematological cancers and solid tumors. Some of these agents are currently in clinical trials.

Human primary NK cells were shown to express SIRPα upon stimulation and bind to CD47. This reduces their killing efficacy for CD47-expressing cells. (See PCT/US20/39220, incorporated by reference herein in its entirety.)

### IV. SUMMARY OF THE INVENTION

The invention provides recombinant CD47 engager receptor proteins comprising an intracellular domain that enhances an immune response in a cell when the CD47 engager receptor protein binds to CD47 on a target cell. The invention also provides cells comprising the CD47 engager receptor proteins. The CD47 engager receptor protein may be a switched Signal Regulatory Protein Alpha (SIRPα) receptor (SIRPα switch receptors) and cells that comprise them (SIRPα switch cells). SIRPα switch receptors recognize CD47 on target tumor cells and transmit an intracellular signal within the SIRPα switch cells that upregulates natural killer cell (NK), T cell, and macrophage activity. The result is increased tumor cell killing.

In some embodiments, the SIRPα switch cells are hypoimmune cells. In other embodiments, the SIRPα switch cells are differentiated somatic cells. In other embodiments, the SIRPα switch cells are hypoimmune pluripotent (HIP) cells. In further embodiments, the HIP cells are blood type O (HIPO), Rhesus factor (Rh) negative (HIP−) or both type O and Rh−(HIPO−). In other embodiments, the SIRPα switch cells have been derived or differentiated from HIP, HIP−, or HIPO− cells. In other embodiments, the SIRPα switch cells comprise an antibody Fc receptor to protect against antibody dependent cellular cytotoxicity (ADCC) or complement dependent cytotoxicity (CDC).

Thus, the invention provides a recombinant CD47 engager receptor protein, comprising an extracellular domain (ECD) that binds to a CD47 protein, a transmembrane domain (TMD), and an intracellular domain (ICD) that enhances an immune response within an immune cell when the ECD recognizes the CD47 protein on a target cell. In some aspects, the CD47 protein comprises at least a 90% sequence identity to SEQ ID NO:6 In preferred aspects, the CD47 sequence comprises the sequence of SEQ ID NO:6. In other aspects, the CD47 protein is a recombinant protein.

In some aspects of the invention, the ECD has at least a 90% sequence identity to SEQ ID NO:2. In preferred aspects, the ECD comprises the sequence of SEQ ID NO:2. In some aspects, the ICD comprises at least a 90% sequence identity to SEQ ID NO:3. In preferred aspects, the ICD comprises the sequence of SEQ ID NO:3. In some aspects, the ICD comprises at least a 90% sequence identity to SEQ ID NO:4 In preferred aspects, the ICD comprises the sequence of SEQ ID NO:4.

The invention provides a recombinant CD47 engager receptor protein as disclosed herein, wherein the TMD comprises at least a 90% sequence identity to SEQ ID NOS:11 or 12. In preferred aspects, the TMD comprises the sequence of SEQ ID NOS:11 or 12.

In other aspects of the invention, the recombinant CD47 engager receptor protein as disclosed herein further comprises a signal peptide. In some aspects, the signal peptide comprises at least a 90% sequence identity to SEQ ID NOS:13 or 14. In preferred aspects, the signal peptide comprises the sequence of SEQ ID NOS:13 or 14.

The invention provides a cell comprising the recombinant CD47 engager receptor proteins as disclosed herein. In some aspects of the invention, the cell is an immune cell. In other aspects, the cell is a natural killer (NK) cell or a T cell. In other aspects, the cell is an NK cell or a macrophage. In other aspects, the NK cell comprises a chimeric antigen receptor (CAR-NK cell). In other aspects, the cell is a T cell. In other aspects, the T cell comprises a chimeric antigen receptor (CAR-T cell).

In some aspects of the invention, the cell disclosed herein, the cell has a reduced or eliminated HLA-I or HLA-II expression. In other aspects, the cell is ABO blood group type O. In other aspects, the cell is Rhesus factor negative (Rh−). In other aspects, the cell has a reduced or eliminated ABO blood group antigen selected from the group consisting of A1, A2, and B. In other aspects, the cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

In some aspects of the invention, the cell is a hypoimmunogenic (HI) cell comprising: an endogenous Major Histocompatibility Complex Class I (HLA-I) function that is reduced when compared to an unmodified parental cell and an endogenous Major Histocompatibility Complex Class II (HLA-II) function that is reduced when compared to the unmodified parental cell.

In some aspects of the invention, the cell comprising the CD47 engager protein further comprises modulated expression of one or more of HLA-I human leukocyte antigens, HLA-II human leukocyte antigens, CD64, CD47, CD38, CCR5, CXCR4, NLRC5, CIITA, B2M, HLA-A, HLA-B, HLA-C, HLA-E, HLA-G, PD-L1, CTLA-4-Ig, CD47, CI-inhibitor, IL-35, RFX-5, RFXAP, RFXANK, NFY-A, NFY-B, NFY-C, IRF-1, OX40, GITR, 4-1BB, CD28, B7-1, B7-2, ICOS, CD27, HVEM, SLAM, CD226, PD1, CTL4, LAG3, TIGIT, TIM3, CD160, BTLA, CD244, CD30, TLT, VISTA, B7-H3, PD-L2, LFA-1, CD2, CD58, ICAM-3, TCRA, TCRB, FOXP3, HELIOS, ST2, PCSK9, APOC3, CD200, FASLG, CLC21, MFGE8, SERPIN B9, TGFβ, CD73, CD39, LAG3, IL1R2, ACKR2, TNFRSF22, TNFRSF23, TNFRS10, DAD1, PVR, and/or IFNγR1 d39 relative to a wild-type stem cell, wherein the engager cell is ABO blood group type O or Rhesus factor negative (Rh−).

In some aspects of the invention, the cell further comprises an elevated expression of an antibody Fc receptor on the cell surface, wherein the Fc receptor helps to evade antibody dependent cellular cytotoxicity (ADCC) or complement mediated cytotoxicity (CDC). In other aspects, the Fc receptor is CD16, CD32, or CD64. In other aspects, the cell is pluripotent. In other aspects, the cell is a hypoimmune pluripotent (HIP) cell. In other aspects, the cell is a hypoimmune pluripotent cell having an ABO blood type O (HIPO). In other aspects, the cell is a hypoimmune pluripotent cell is Rh factor negative (HIP−). In preferred aspects, the cell is a hypoimmune pluripotent cell having an ABO blood type O and is Rh factor negative (HIPO−).

In some aspects of the invention, the cell is a pluripotent (PSC) cell, induced PSC (iPSC), or an embryonic stem cell (ESC). In other aspects, the cell is differentiated from a pluripotent cell.

The invention provides a pharmaceutical composition, comprising the cells as disclosed herein and a pharmaceutically-acceptable carrier.

The invention provides a medicament, comprising the SIRP-α switch receptor cells as disclosed herein and a pharmaceutically-acceptable carrier.

The invention provides a method of treating a disease in a subject, comprising transplanting the cells as disclosed herein. In some aspects, the disease is cancer. In other aspects, the disease is leukemia, lymphoma, myeloma, a solid tumor, or a liquid tumor.

The invention provides a use of the cells as disclosed herein for preparing a pharmaceutical composition for treating a disease in a subject. In some aspects, the disease is cancer. In other aspects, the disease is leukemia, lymphoma, myeloma, a solid tumor, or a liquid tumor.

The invention provides a use of the cells as disclosed herein for treating a disease in a subject. In some aspects, the disease is cancer. In other aspects, the disease is leukemia, lymphoma, myeloma, a solid tumor, or a liquid tumor.

### V. BRIEF DESCRIPTION OF THE DRAWINGS

FIG. 1: A great variety of cancer cells show high expression of CD47, which conveys some immune protection. Natural NK cells express the CD47 receptor, SIRPα, in inflammatory environments. CD47 transmits a strong inhibitory signal to the NK cell upon CD47 binding. The NK cell is inhibited and the cancer evades killing. SIRPα switch receptors turn the inhibitory SIRPα signal into an activating signal. Upon CD47 binding, the switch receptors transmit intracellular signaling through activating domains such as those from CD16 or CD28. This activates NK cells and enhances their cytotoxicity.

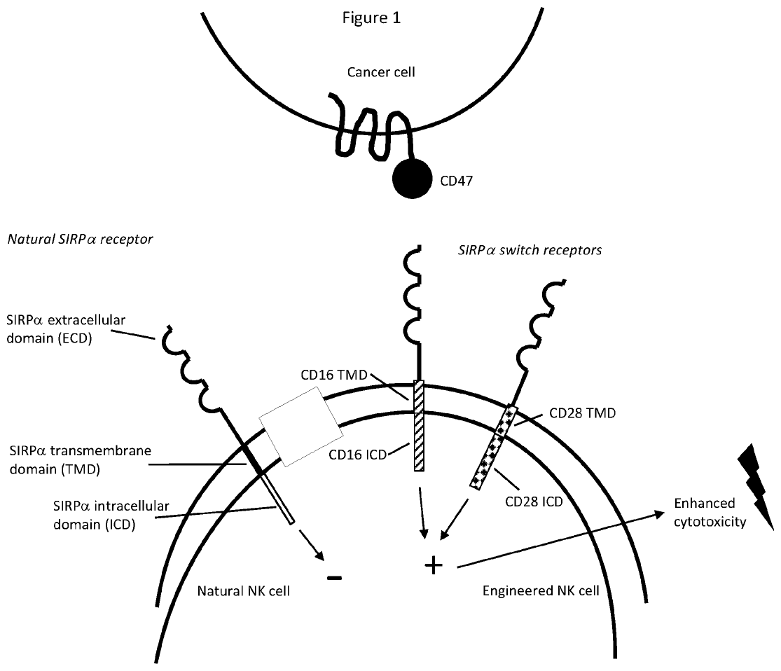

FIGS. 2A and 2B: In vitro killing capacity of primary human NK cells and primary human NK cells engineered to express the SIRPα-CD16 switch receptor. K562 target cells in the presence of IL-2 were similarly killed by both NKs and NK (SIRPα-CD16 switch)(FIG. 2A). K562 target cells overexpressing CD47 (K562-CD47OV), however, were killed far more aggressively by NK (SIRPα-CD16 switch) cells (FIG. 2B).

FIGS. 3A and 3B: In vitro killing capacity of primary human NK cells and primary human NK cells engineered to express the SIRPα-CD28 switch receptor. K562 target cells in the presence of IL-2 were similarly killed by both NKs and NK (SIRPα-CD28 switch)(FIG. 3A). K562 target cells overexpressing CD47 (K562-CD47OV), however, were killed far more aggressively by NK (SIRPα-CD28 switch) cells (FIG. 3B).

FIG. 4: In vitro tumor killing assays by primary human peripheral blood NK cells. NK cells effectively killed K562 targets on the xCelligence impedance platform (upper row) but fail to kill K562 target cells overexpressing CD47 (K562-CD47OV) (lower row).

FIG. 5: In vitro tumor killing assays by human SIRPα-CD16 switch NK cells. Primary human NK cells expressing the SIRPα-CD16 switch receptor effectively killed both K562 targets (upper row) and K562 target cells overexpressing CD47 (K562-CD47OV) (lower row).

FIG. 6: In vitro tumor killing assays by human SIRPα-CD28 switch NK cells. Primary human NK cells expressing the SIRPα-CD28 switch receptor effectively killed both K562 targets (upper row) and K562 target cells overexpressing CD47 (K562-CD47OV) (lower row).

### VI. DETAILED DESCRIPTION OF THE INVENTION

The invention provides a switched Signal Regulatory Protein Alpha (SIRPα) receptor function (SIRPα switch receptors) and cells that comprise them (SIRPα switch cells). SIRPα switch receptors recognize CD47 on target tumor cells and transmit an intracellular signal within the SIRPα switch cells that upregulates natural killer cell (NK), T cell, and macrophage activity. The result is increased tumor cell killing. In some embodiments, the SIRPα switch cells are hypoimmune cells. In other embodiments, the SIRPα switch cells are differentiated somatic cells. In other embodiments, the SIRPα switch cells are hypoimmune pluripotent (HIP) cells. In further embodiments, the HIP cells are blood type O (HIPO), Rhesus factor (Rh) negative (HIP−) or both type O and Rh− (HIPO−). In other embodiments, the SIRPα switch cells have been derived or differentiated from HIP, HIP−, or HIPO− cells. In other embodiments, the SIRPα switch cells comprise an antibody Fc receptor to protect against antibody dependent cellular cytotoxicity (ADCC) or complement dependent cytotoxicity (CDC).

The invention provides, in part, the use of a newly-discovered inhibitory immune checkpoint in NK cells involving SIRPα. Upon ligation of its natural ligand, CD47 or a synthetic engager molecule, NK cell SIRPα delivers an inhibitory signal and mitigates cytotoxicity. Since many tumor cells have strong CD47 expression, they inhibit NK cells and evade cell killing. NK cells with reduced SIRPα expression have stronger cytotoxic effector function against tumors expressing CD47 than unmodified NK cells (WO2020263880, incorporated by reference herein in its entirety). Building upon this technology, in addition to minimizing the inhibitory signaling of wild-type SIRPα, SIRPα switch receptors are introduced that transmit activating signals for NK cell killing upon recognition of CD47 on tumor cells. This is done by replacing the transmembrane and intracellular signaling domains of SIRPα with the transmembrane and intracellular signaling domains of activating receptors such as CD16 or CD28. The result is a modified NK cell that has increased killing capacity against targets expressing SIRPα ligands.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic” cell or “HI” cell herein is meant a cell that gives rise to a reduced immunological rejection response when transferred into an allogeneic host. In preferred embodiments, HI cells do not give rise to an immune response. Thus, “hypo-immunogenic” refers to a significantly reduced or eliminated immune response when compared to the immune response of a parental (i.e. “wt”) cell prior to immunoengineering.

By “hypo-immunogenic cell O−” “hypo-immunogenic ORh−” cell or “HIO−” cell herein is meant a HI cell that is also ABO blood group O and Rhesus Factor Rh−. HIO− cells may have been generated from O− cells, enzymatically modified to be O−, or genetically engineered to be O−.

By “HLA” or “human leukocyte antigen” complex herein is meant a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or non-human (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences. “β-2 microglobulin” or “β2M” or “β2M” protein refers to the human 02M protein that has the amino acid and nucleic acid sequences shown below; the human gene has accession number RefSeq NM_004048.4.

“CD47 protein” protein refers to the human CD47 protein that has the amino acid and nucleic acid sequences shown below; the human gene has accession number RefSeq NM_001777.4.

CD47 expression on engineered cells has been shown to provide protection against innate immune cell killing and phagocytosis (Deuse T. Nat Biotechnol. 2019 March; 37:252-258). However, upon ligation of its ligand SIRPα, CD47 can initiate downstream signaling in the engineered cell with potentially unwanted perturbations of its physiology. To only achieve protection against immune cell killing, novel hypoimmune strategies separate the extracellular SIRPα-binding function from intracellular signaling in the engineered cell. This invention provides SIRPα switch receptors or NK cells bearing them that target tumor cells or other cells expressing CD47 or a synthetic SIRPα engager and exert killing. The other cells might include engineered hypoimmune cells that may be killed as a safety strategy by the cells of the invention.

The invention provides a SIRPα switch receptor. It is a fusion protein that is expressed on an engineered immune cell such as an NK cell. It is designed to bind to CD47 on a tumor cell or other cells such as hypoimmune cells. The effector immune cell can be any immune cell expressing the SIRPα switch receptor and can be from the myeloid lineage (e.g. monocytes, macrophages, or polymorphonuclear cells) as well as the lymphoid lineage (e.g. T cells, B cells, or NK cells).

The fusion proteins provided herein comprise an extracellular domain (ECD), a transmembrane domain (TMD), and an immune-activating intracellular domain (ICD). Examples of ICDs include those of CD16, CD28, CD28, CD16, 4-1BB, ICOS, OX40, CRTAM, CXADR, CD7, NTB-A, CD244, CD200R, NKR-PIA, GITR, CD30, CD40, CD96, CD2, TIM1, NKG2D, CD72, DC-SIGN, Siglec-3, TACI, BAFF-R, BCMA, CD300f, CD300a, and CD3z.

In some aspects, one or more residues of the SIRPα switch receptor sequence are altered to modify binding to achieve a more favored on-rate of binding to CD47, a more favored off-rate of binding, or both, such that an optimized binding is achieved.

In other aspects, the ECD contains linker regions or hinges connecting the sequences provided with either the TMD or with each other. In other aspects, modifications are made within one or more of the linker regions or hinge regions so long as these modifications do not eliminate the binding affinity of the fusion protein with CD47.

In some aspects, an ECD has a contiguous sequence of at least about 10 amino acids from SEQ ID NO:1, at least about 15 amino acids, at least about 20 amino acids, at least about 25 amino acids, at least about 30 amino acids, up to the complete ECD region. In other aspects, the ECD comprises a protein having the sequence of SEQ ID NO:2. In other aspects, ECDs also include sequences that differ by up to 1, 2, 3, 4, 5, 6 or more amino acids as compared to the amino acid sequence set forth in SEQ ID NO:2. In other aspects, an ECD is a protein having at least about an 80%, 85%, 90%, 95%, or about 99% sequence identity to the amino acid sequence set forth in SEQ ID NOS:2.

In other aspects, an ECD has anti-CD47 antibody sequences. Anti-CD47 antibodies are known in the art and are disclosed, eg, in WO2011143624 which is incorporated by reference herein in its entirety.

Generally, the transmembrane domain (TMD) of the SIRPα switch receptor protein is not limited to a specific TMD sequence. Preferably, the TMD allows stable anchorage of the fusion protein in the membrane of a cell expressing the SIRPα switch receptor. It further allows binding of the ECD to CD47.

TMDs extend across the cell membrane lipid bilayer as a single a helix, as multiple a helices, or as a rolled-up β sheet. Some of these “single-pass” and “multipass” proteins have a covalently attached fatty acid chain inserted in the cytosolic lipid monolayer. Other membrane proteins are exposed at only one side of the membrane. Some of these are anchored to the cytosolic surface by an amphipathic a helix that partitions into the cytosolic monolayer of the lipid bilayer through the hydrophobic face of the helix. Others are attached to the bilayer solely by a covalently attached lipid chain—either a fatty acid chain or a prenyl group—in the cytosolic monolayer or, via an oligosaccharide linker, to phosphatidylinositol in the noncytosolic monolayer. (Alberts B, Johnson A, Lewis J, et al., Molecular Biology of the Cell. 4th edition. New York: Garland Science; ISBN-10: 0-8153-3218-1 (2002)).

In some aspects, an exemplary TMD of the SIRPα switch receptor is from CD85f, CD349, CD284, CD261, CD172b, CD277, CD186, CD156c, CD304, CD254, CD263, CD267, CD337, CD170, CD283, CD133, CD327, CD205, CD232, CD282, CD16b, CD85i, CD85a, CD85c, CD275, CD108, CD358, CD335, CD218b, CD355, CD336, CD160, CD25, CD4, CD8a, CD235a, CD233, CD230, CD90, CD74, CD3d, CD340, CD236, CD61, CD18, CD54, CD29, CD1a, CD5, CD220, CD2, CD66e, CD51, CD141, CD115, CD42b, CD221, CD271, CD55, CD243, CD98, CD10, CD41, CD14, CD45, CD228, CD16a, CD49e, CD126, CD63, CD48, CD7, CD140b, CD3g, CD117, CD28, CD8b, CD37, CD11b, CD107a, CD331, CD222, CD20, CD79a, CD64, CD32, CD143, CD324, CD42c, CD107b, CD56, CD102, CD49d, CD66a, CD142, CD59, CD62L, CD121a, CD122, CD13, CD155, CD119, CD19, CD116, CD46, CD1e, CD1d, CD227, CD44, CD62P, CD104, CD43, CD140a, CD31, CD152, CD326, CD62E, CD36, CD127, CD49b, CD105, CD35, CD223, CD138, CD325, CD58, CD106, CD53, CD120a, CD224, CD21, CD33, CD22, CD120b, CD11a, CD11c, CD363, CD73, CD88, CD204, CD332, CD9, CD203a, CD334, CD333, CD206, CD49f, CD238, CD252, CD89, CD124, CD181, CD182, CD24, CD95, CD40, CD49c, CD159a, CD159c, CD314, CD27, CD123, CD26, CD82, CD121b, CD34, CD38, CD30, CD1b, CD1c, CD154, CD6, CD52, CD132, CD32, CD66b, CD171, CD191, CD197, CD185, CD131, CD50, CD70, CD153, CD144, CD80, CD362, CD68, CD361, CD147, CD309, CD135, CD292, CD103, CD130, CD42d, CD66d, CD66c, CD96, CD110, CD79b, CD200, CD192, CD231, CD86, CD212, CD118, CD146, CD134, CD158a, CD158b1, CD158b2, CD158e, CD158k, CD158j, CD158i, CD178, CD295, CD151, CD97, CD183, CD39, CD239, CD193, CD194, CD195, CD196, CDw198, CDw199, CD296, CD298, CD49a, CD322, CD85g, CD184, CD172a, CD156a, CD339, CD156b, CD213a1, CD129, CD83, CD125, CD241, CD269, CD202b, CD87, CD164, CD136, CD137, CD249, CD69, CD91, CDw210b, CD167a, CD300c, CD47, CD157, CD317, CD148, CD161, CD215, CD150, CD11d, CD218a, CD210, CD166, CD162, CD213a2, CD242, CD158g, CD158h, CD279, CD111, CD281, CD226, CD234, CD167b, CD300e, CD276, CD305, CD300g, CD300d, CD109, CD272, CD163, CD302, CD158f1, CD85h, CD85d, CD177, CD158z, CD158f2, CD85j, CD300f, CD92, CD351, CD112, CD100, CD270, CD101, CD297, CD316, CD352, CD217, CD307b, CD307a, CD307c, CD307d, CD307e, CD114, CD180, CD158d, CD273, CD290, CD244, CD169, CD299, CD318, CD360, CD229, CD248, CD354, CD320, CD93, CD319, CD113, CD163b, CD289, CD288, CD329, CD274, CD353, CD172g, CD315, CD280, CD264, CD300a, CD312, CD84, CD344, CD350, CD246, CD201, CD338, CD208, CD257, CD328, CD286, CD357, CD294, CD321, CD265, CD278, ITGA7, ITGA8, ITGA9, ITGA10, ITGA11, CD51, CD41, CD29, CD18, CD61, CD104, and PDGF.

“CIITA protein” protein refers to the human CIITA protein that has the amino acid and nucleic acid sequences shown below; the human gene has the RefSeq accession number NM_000246.4.

By “wild type” in the context of a cell means a cell found in nature. However, in the context of a natural killer (NK) cell, as used herein, it also means that the cell may contain nucleic acid changes resulting in immortality but did not undergo the gene editing procedures of the invention to achieve hypo-immunogenicity.

By “syngeneic” herein refers to the genetic similarity or identity of a host organism and a cellular transplant where there is immunological compatibility; e.g. no immune response is generated.

By “allogeneic” herein refers to the genetic dissimilarity of a host organism and a cellular transplant where an immune response is generated.

By “B2M−/−” herein is meant that a diploid cell has had the B2M gene inactivated in both chromosomes. As described herein, this can be done in a variety of ways.

By “CIITA−/−” herein is meant that a diploid cell has had the CIITA gene inactivated in both chromosomes. As described herein, this can be done in a variety of ways.

By “CD47 tg,” “CD47 transgene,” or “CD47+” herein is meant that the host cell expresses CD47, in some cases by having at least one additional copy of the CD47 gene.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at Ph 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (Ph 6.8), 0.1% sodium pyrophosphate, 5×Denhardt's solution, sonicated salmon sperm DNA (50 μl/m1), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, a “pharmaceutically acceptable carrier” or “therapeutic effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil. A pharmaceutically acceptable carrier can contain physiologically acceptable compounds that act, for example, to stabilize or to increase the absorption of specific inhibitor, for example, carbohydrates, such as glucose, sucrose or dextrans, antioxidants such as ascorbic acid or glutathione, chelating agents, low molecular weight proteins or other stabilizers or excipients.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In some embodiments, an insertion, deletion, substitution, or other type of amino acid change in a SIRPα, CD47, CD16, CD32, CD64, HSVtk, EC-CD, or iCasp9 variant polypeptide is prepared according to the methods described herein and known in the art. Such modifications differentiate them from the corresponding parent that has not been modified according to the methods described herein, such as wild-type proteins, naturally occurring mutant proteins, or another engineered proteins that do not include the modifications of such variant polypeptides. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

### VII. CELLS OF THE INVENTION

The invention provides a SIRPα receptor function (SIRPα switch receptors) and cells that comprise them (SIRPα switch cells). SIRPα switch receptors recognize CD47 on target tumor cells and transmit an intracellular signal within the SIRPα switch cells that upregulates natural killer cell (NK) activity. The result is increased tumor cell killing. In some embodiments, the SIRPα switch cells are hypoimmune cells. In other embodiments, the SIRPα switch cells are differentiated somatic cells. In other embodiments, the SIRPα switch cells are hypoimmune pluripotent (HIP) cells. In further embodiments, the HIP cells are blood type O (HIPO), Rhesus factor (Rh) negative (HIP−) or both type O and Rh− (HIPO−). In other embodiments, the SIRPα switch cells have been derived or differentiated from HIP, HIP−, or HIPO− cells. In other embodiments, the SIRPα switch cells comprise an antibody Fc receptor to protect against antibody dependent cellular cytotoxicity (ADCC) or complement dependent cytotoxicity (CDC).

The invention provides compositions and methodologies for generating a SIRPα switch cell. In some aspects, the cells are hypoimmune cells. In other aspects, the cells are differentiated somatic cells. In other aspects, the cells are pluripotent cells such as HIP cells, HIP− cells, HIPO− cells. In other aspects, the SIRPα switch cells are pluripotent (PSC) cells suitable for transplantion and/or differentiation. The PSC cells include induced PSCs (iPSC) or embryonic stem cells (ESC). In other aspects, the cells are of particular tissue types and have differentiated from the aforementioned SIRPα switch cells. By way of example, the differentiated SIRPα switch cells may be chimeric antigen receptor (CAR) cells, such as CAR-T cells, CAR-NK cells, and other engineered cell populations. See WO2018/132783, WO2020/018620, WO2020/018615, PCT/US2020/032272, and U.S. patent application Ser. Nos. 16/870,959, 16/870,960, incorporated by reference herein in their entirety.

The invention provides SIRPα switch cells having SIRPα switch receptor proteins that interact with CD47 to enhance tumor cell killing and innate immunity. In other embodiments, a CD47 engager protein is used having anti-CD47 antibody sequences fused to an immune-enhancing intracellular domain such as that of CD16 or CD28. In some embodiments, the anti-CD47 antibody sequences are tethered via a fragment crystallizable (Fc) portion to a cell-surface CD. In other embodiments, the antigen-binding portion of the anti-CD47 antibody (scFv) are bound to the cell surface via a transmembrane domain (TMD). In preferred embodiments, the TMD comprises one or more α-helices. In other preferred embodiments, the TMD is from a 7 transmembrane protein (7TM). In other preferred embodiments, the TMD is from an immunoglobulin cell-surface protein. In more preferred embodiments, the immunoglobulin cell-surface protein is an antibody, receptor, ligand, or adhesion protein. In some embodiments, the CD47 engager cell results from a CD47 engager protein anchored onto the cell surface.

CD47 engager protein, including SIRPα switch protein, expression may be accomplished in several ways as will be appreciated by those in the art using “knock in” or transgenic technologies. In some cases, SIRPα switch protein expression results from one or more transgenes.

Accordingly, in some embodiments, one or more copies of a SIRPα switch protein expression gene is added to the SIRPα switch receptor cells under the control of an inducible or constitutive promoter, with the latter being preferred. In some embodiments, a lentiviral construct is employed as described herein or known in the art. The genes may integrate into the genome of the host cell under the control of a suitable promoter as is known in the art.

In some embodiments, the expression of the gene can be increased by altering the regulatory sequences of an endogenous gene locus, for example, by exchanging the endogenous promoter for a constitutive promoter or for a different inducible promoter. This can generally be done using known techniques such as CRISPR.

Once altered, the presence of sufficient SIRPα switch protein expression can be assayed using known techniques such as those described in the Examples, such as Western blots, ELISA assays or FACS assays using appropriate antibodies. In general, “sufficiency” in this context means an increase in SIRPα switch protein expression on the cell surface that activates NK cell killing.

Also within the scope of the invention are polypeptides that are antibodies. The term antibody is meant to include monoclonal antibodies, polyclonal antibodies, humanized antibodies, antibody fragments (e.g., Fc domains), Fab fragments, single chain antibodies, bi- or multi-specific antibodies, Llama antibodies, nano-bodies, diabodies, affibodies, Fv, Fab, F(ab′)2, Fab′, scFv, scFv-Fc, and the like. Also included in the term are antibody-fusion proteins, such as Ig chimeras. Preferred antibodies include humanized or fully human monoclonal antibodies or fragments thereof.

The terms “antibody” and “immunoglobulin” may include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region. “Antibody fragments” comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments. The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Antibodies that bind specifically to an antigen have a high affinity for that antigen. Antibody affinities may be measured by a dissociation constant (Kd). In certain embodiments, an antibody provided herein has a dissociation constant (Kd) of equal to or less than about 100 nM, 10 nM, 1 nM, 0.1 nM, 0.01 nM, or 0.001 nM (e.g. 10⁻⁷ M or less, from 10⁻⁷ M to 10⁻¹³ M, from 10⁻⁸ M to 10⁻¹³ Mor from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with, e.g., immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (Kₒₙ) and dissociation rates (K_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Other coupling chemistries for the target antigen to the chip surface (e.g., streptavidin/biotin, hydrophobic interaction, or disulfide chemistry) are also readily available instead of the amine coupling methodology (CM5 chip) described above, as will be understood by one of ordinary skill in the art.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al, Nature, 256: 495 (1975); Harlow et al, Antibodies: A Laboratory Manual, (Cold Spring Harbor Laboratory Press, 2nd ed. 1988); Hammerling et al., in: Monoclonal Antibodies and T-Cell Hybridomas pp. 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995). The above patents, publications, and references are incorporated by reference in their entirety.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23: 1035-1038 (1995); Hurle and Gross, Curr. Op. Biotech. 5:428-433 (1994). The foregoing references are incorporated by reference in their entirety.

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol, 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor, J. Immunol, 133: 3001 (1984); Brodeur et al., Monoclonal Antibody Production Techniques and Applications, pp. 55-93 (Marcel Dekker, Inc., New York, 1987); and Boerner et al., J. Immunol, 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

### A. Methodologies for Genetic Alterations

The invention includes methods of modifying nucleic acid sequences within cells or in cell-free conditions to generate SIRPα switch cells. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the modified cells of the invention, as well as the engineering them to become hypo-immunogenic as outlined herein.

In general, these techniques can be used individually or in combination. For example, in the generation of the SIRPα switch receptor cells, CRISPR may be used to express SIRPα switch proteins such as anti-CD47 immunoglobulins. In another example, viral techniques (e.g. lentivirus) are used to express SIRPα, switch proteins.

a. CRISPR Technologies

In one embodiment, the cells are manipulated using clustered regularly interspaced short palindromic repeats)/Cas (“CRISPR”) technologies as is known in the art. CRISPR can be used to generate the SIRPα switch receptor cells. There are a large number of techniques based on CRISPR, see for example Doudna and Charpentier, Science doi:10.1126/science.1258096, hereby incorporated by reference. CRISPR techniques and kits are sold commercially.

b. TALEN Technologies

In some embodiments, the cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

c. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

d. Viral Based Technologies

There are a wide variety of viral techniques that can be used to generate some embodiments of the SIRPα switch cells of the invention including, but not limited to, the use of retroviral vectors, lentiviral vectors, adenovirus vectors and Sendai viral vectors. Episomal vectors used in the generation of the cells are described below.

For all of these technologies, well known recombinant techniques are used, to generate recombinant nucleic acids as outlined herein. In certain embodiments, the recombinant nucleic acids that encode a SIRPα switch protein, e.g. an anti-CD47 immunoglobulin, may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, the early promoter of human Cytomegalovirus (CMV), the eukaryotic translation elongation factor 1α (EF-1α), and the chicken β-Actin promoter coupled with CMV early enhancer (CAG). Examples of other heterologous mammalian promoters are the actin, immunoglobulin or heat shock promoter(s).

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

In some embodiments, the SIRPα switch cells are derived from stem cells.

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells (ESC) and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics.

As described herein, cells do not need to pass through pluripotency to be reprogrammed into endodermal progenitor cells and/or hepatocytes.

### B. Generation of Hypo-Immunogenic SIRPα Switch Cells

Generating HI cells is done with as few as three genetic changes, resulting in minimal disruption of cellular activity but conferring immunosilencing to the cells. The techniques are disclosed in WO2018/132783, WO2020/018620, WO2020/018615, PCT/US2020/032272, and U.S. patent application Ser. Nos. 16/870,959, 16/870,960, incorporated by reference herein in their entirety. The techniques are discussed briefly below.

As discussed herein, one embodiment utilizes a reduction or elimination in the protein activity of MHC I and II (HLA I and II when the cells are human). This can be done by altering genes encoding their components. In one embodiment, the coding region or regulatory sequences of the gene are disrupted using CRISPR. In another embodiment, gene translation is reduced using interfering RNA technologies. Another embodiment is a change in a gene that regulates susceptibility to macrophage phagocytosis. This may be a “knock in” of a gene using viral technologies.

### 1. HLA-I Reduction

The HI SIRPα switch cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the SIRPα switch cells can be measured using techniques known in the art and as described below; for example, FACS techniques using labeled antibodies that bind the HLA complex; for example, using commercially available HLA-A,B,C antibodies that bind to the the alpha chain of the human major histocompatibility HLA Class I antigens.

a. B2M Alteration

In one embodiment, the reduction in HLA-I activity is done by disrupting the expression of the β-2 microglobulin gene in the HI SIRPα switch cell, as disclosed herein. This alteration is generally referred to herein as a gene “knock out”, and in the cells of the invention it is done on both alleles in the host cell. Generally the techniques to do both disruptions is the same.

A particularly useful embodiment uses CRISPR technology to disrupt the gene. Another embodiment uses programmable transcriptional memory by CRISPR-based epigenome editing (Nunez J K, Cell. 184:2503-2519 (2021), incorporated by reference herein in its entirety). In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected SIRPα switch cell cultures are dissociated to single cells. Single cells are expanded to full-size colonies and tested for CRISPR edit by screening for presence of aberrant sequence from the CRISPR cleavage site. Clones with deletions in both alleles are picked. Such clones did not express B2M as demonstrated by PCR and did not express HLA-I as demonstrated by FACS analysis.

Assays to test whether the B2M gene has been inactivated are known and described herein. In one embodiment, the assay is a Western blot of cells lysates probed with antibodies to the B2M protein. In another embodiment, reverse transcriptase polymerase chain reactions (rt-PCR) confirms the presence of the inactivating alteration.

In addition, the cells can be tested to confirm that the HLA I complex is not expressed on the cell surface. This may be assayed by FACS analysis using antibodies to one or more HLA cell surface components as discussed above.

### 2. HLA-II Reduction

In some embodiments, in addition to a reduction in HLA I, the HI SIRPα switch cells of the invention may also lack MHC II function (HLA II from human-derived cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II (HLA II) function in the SIRPα switch cells or their derivatives can be measured using techniques known in the art such as Western blotting using antibodies to the protein, FACS techniques, rt-PCR techniques, etc.

a. CIITA Alteration

In one embodiment, the reduction in HLA-II activity is done by disrupting the expression of the CIITA gene in the SIRPα switch cell, as shown herein. This alteration is generally referred to herein as a gene “knock out”, and in the SIRPα switch cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, reverse transcriptase polymerase chain reactions (rt-PCR) confirms the presence of the inactivating alteration.

In addition, the cells can be tested to confirm that the HLA II complex is not expressed on the cell surface. Again, this assay is done as is known in the art. Exemplary analyses include Western Blots or FACS analysis using commercial antibodies that bind to human HLA Class II HLA-DR, DP and most DQ antigens as outlined below.

A particularly useful embodiment uses CRISPR technology to disrupt the CIITA gene. CRISPRs ae designed to target the coding sequence of the CIITA gene, an essential transcription factor for all MHC II molecules. After gene editing, the transfected cell cultures are dissociated into single cells. They are expanded to full-size colonies and tested for successful CRISPR editing by screening for the presence of an aberrant sequence from the CRISPR cleavage site. Clones with deletions that do not express CIITA are determined by PCR and may be shown not to express MHC II/HLA-II by FACS analysis. Another embodiment uses programmable transcriptional memory by CRISPR-based epigenome editing.

### 3. Blood Type O Rh Negative Cells

Blood products can be classified into different groups according to the presence or absence of antigens on the surface of every red blood cell in a person's body (ABO Blood Type). The A, B, AB, and A1 antigens are determined by the sequence of oligosaccharides on the glycoproteins of erythrocytes. The genes in the blood group antigen group provide instructions for making antigen proteins. Blood group antigen proteins serve a variety of functions within the cell membrane of red blood cells. These protein functions include transporting other proteins and molecules into and out of the cell, maintaining cell structure, attaching to other cells and molecules, and participating in chemical reactions.

The Rhesus Factor (Rh) blood group is the second most important blood group system, after the ABO blood group system. The Rh blood group system consists of 49 defined blood group antigens, among which five antigens, D, C, c, E, and e, are the most important. Rh(D) status of an individual is normally described with a positive or negative suffix after the ABO type. The terms “Rh factor,” “Rh positive,” and “Rh negative” refer to the Rh(D) antigen only. Antibodies to Rh antigens can be involved in hemolytic transfusion reactions and antibodies to the Rh(D) and Rh(c) antigens confer significant risk of hemolytic disease of the fetus and newborn. ABO antibodies develop in early life in every human. However, rhesus antibodies in Rh− humans develop only when the person is sensitized. This occurs by giving birth to a rh+ baby or by receiving an Rh+ blood transfusion.

This invention provides SIRPα switch cells having an ABO blood type O and/or Rhesus Factor negative (O−) populations of pluripotent (PSCO−) cells suitable for transplantion and/or differentiation. The PSCO− cells include induced iPSCs (iPSCO−), embryonic ESCs (ESCO−), and cells differentiated from those cells, including O− endothelial cells, O− cardiomyocytes, O− hepatocytes, O− dopaminergic neurons, O− pancreatic islet cells, O− retinal pigment endothelium cells, and other O− cell types used for transplantation and medical therapies. These would include O− chimeric antigen receptor (CAR) cells, such as CAR-T cells, CAR-NK cells, and other engineered cell populations. In some embodiments, the cells are not hematopoietics stem cells. The invention further provides universally acceptable “off-the-shelf” ESCO-s and PSCO-s and derivatives thereof for generating or regenerating specific tissues and organs.

Another aspect of the invention provides methods of generating populations of PSCO−, iPSCO−, ESCO− and other O− cells for transplantation. The invention also provides methods of treating diseases, disorders, and conditions that benefit from the transplantation of pluripotent or differentiated cells.

In some embodiments of the invention, the ABO blood group type O results from a reduced ABO blood group protein expression. In other aspects, the ABO blood group is endogenously type O. In some aspects of the invention, the HIPO− cell has an ABO blood group type O that results from a disruption in human Exon 7 of the ABO gene. In some embodiments, both alleles of Exon 7 of the ABO gene are disrupted. In some embodiments, the disruption in both alleles of Exon 7 of the ABO gene results from a Clustered Regularly Interspaced Short Palindromic Repeats (CRISPR)/Cas9 reaction that disrupts both of the alleles. Another embodiment uses programmable transcriptional memory by CRISPR-based epigenome editing to inactivate this gene.

In other aspects, the ABO blood group type O results from an enzymatic modification of an ABO gene product on a surface of the cell. In a preferred aspect, the enzymatic modification removes a carbohydrate from the ABO gene product. In another preferred aspect, the enzymatic modification removes a carbohydrate from an ABO A1 antigen, A2 antigen, or B antigen.

In some embodiments of the invention, the Rh blood group is endogenously type Rh−. In another aspect, the Rh− blood group results from reducing or eliminating Rh protein expression. In another aspect, the type Rh− results from disrupting the gene encoding Rh C antigen, Rh E antigen, Kell K antigen (KEL), Duffy (FY) Fya antigen, Duffy Fy3 antigen, Kidd (JK) Jkb antigen, or/and or Kidd SLC14A1. In some embodiments the disruption results from a CRISPR/Cas9 reaction that disrupts both alleles of the gene encoding Rh C antigen, Rh E antigen, Kell K antigen (KEL), Duffy (FY) Fya antigen, Duffy Fy3 antigen, Kidd (JK) Jkb antigen, or/and or Kidd SLC14A1.

In some embodiments of the invention, the O− cells (e.g., PSCO−, iPSCO−, ESCO− and cells derived therefrom) of the invention are of mammalian origin, for example, human, bovine, porcine, chicken, turkey, horse, sheep, goat, donkey, mule, duck, goose, buffalo, camel, yak, llama, alpaca, mouse, rat, dog, cat, hamster, or guinea pig origin.

In a specific embodiment, the invention provides hypoimmune SIRPα switch cells with an ABO blood type O Rhesus Factor negative (HIPO−) cells that evade rejection by the host allogeneic immune system and avoid blood antigen type rejection. In some embodiments, the HIPO− cells are engineered to reduce or eliminate HLA-I and HLA-II expression, increase expression of an endogenous protein that reduces the susceptibility of the pluripotent cell to macrophage phagocytosis, and comprise a universal blood group O Rh− (“O−”) blood type. The universal blood type may be achieved by eliminating ABO blood group A and B antigens and Rh factor expression, or by starting with an O− cell line. These novel HIPO− cells evade host immune rejection because they have an impaired antigen presentation capacity, protection from innate immune clearance, and lack blood group rejection.

### 4. Suicide Genes

In some embodiments, the invention provides HI SIRPα, switch cells that comprise a “suicide gene” or “suicide switch”. These are incorporated to function as a “safety switch” that can cause the death of the cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., CellRes. 8:73-8 (1998), both incorporated herein by reference in their entirety.

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N. Engl. J. Med 365; 18 (2011); Tey et al., Biol. Blood Marrow Transplant. 13:913-924 (2007), each of which are incorporated by reference herein in their entirety.)

### 5. Fc Sequestration

If an antibody binds to an unprotected cell via its Fab regions, the Fc can be bound by NK cells (mostly via their CD16 receptor), macrophages (mostly via CD16, CD32, or CD64), B-cells (mostly via CD32), or granulocytes (mostly via CD16, CD32, or CD64). These can mediate antibody-dependent cellular cytotoxicity (ADCC). If complement binds to the Fc, it can cause complement dependent cytotoxicity (CDC).

In some embodiments, the SIRPα switch cells of the invention comprise elevated levels of receptors that recognize the Fc portion of IgG. Receptors that recognize the Fc portion of IgG are divided into four different classes: FcγRI (CD64), FcγRII (CD32), FcγRIII (CD16), and FcγRIV. This reduces the propensity for the cell transplant recipient's immune system to reject allogeneic material. The cells expressing elevated CD16, CD32, or CD64 evade ADCC or CDC. Fc Sequestration is disclosed in WO2021076427, incorporated by reference herein in its entirety.

### 6. Assays for HI Phenotypes

Once the HI cells have been generated, they may be assayed for their hypo-immunogenicity as is generally described herein.

For example, hypo-immunogenicity is assayed using a number of techniques. One exemplary technique includes transplantation into allogeneic hosts and monitoring for HI SIRPα switch receptor cell survival. The cells may be transduced to express luciferase and can then be followed using bioluminescence imaging. Similarly, the T cell or B cell response of the host animal to the HI SIRPα, switch receptor cells are tested to confirm that they do not cause an immune reaction in the host animal. T cell function is assessed by Elispot, Elisa, FACS, PCR, or mass cytometry (CYTOF). B cell response or antibody response is assessed using FACS or luminex. Additionally, or alternatively, the cells may be assayed for their ability to avoid innate immune responses, e.g. NK cell killing. NK cell cytolytic activity is assessed in vitro or in vivo using techniques known in the art.

### C. Generation of Hypoimmune (HI) O− SIRPα Switch Receptor Cells

In some aspects of the invention, the SIRPα switch receptor cells generated as above will already be ABO blood group O and Rh factor negative (−) cells because the process will have started with NK cells having an O− blood type.

Other aspects of the invention involve the enzymatic conversion of A and B antigens. In preferred aspects, the B antigen is converted to O using an enzyme. In more preferred aspects, the enzyme is an α-galactosidase. This enzyme eliminates the terminal galactose residue of the B antigen. Other aspects of the invention involve the enzymatic conversion of A antigen to O. In preferred aspects, the A antigen is converted to O using an α-N-acetylgalactosaminidase. Enzymatic conversion is discussed, e.g., in Olsson et al., Transfusion Clinique et Biologique 11:33-39 (2004); U.S. Pat. Nos. 4,427,777, 5,606,042, 5,633,130, 5,731,426, 6,184,017, 4,609,627, and 5,606,042; and Int'l Pub. No. WO9923210, each of which are incorporated by reference herein in their entirety.

Other embodiments of the invention involve genetically engineering the cells by knocking out the ABO gene Exon 7 or silencing the SLC14A1 (JK) gene. Other embodiments of the invention involve knocking out the C and E antigens of the Rh blood group system (RH), K in the Kell system (KEL), Fya and Fy3 in the Duffy system (FY), Jkb in the Kidd system (JK), or U and S in the MNS blood group system. Any knockout methodology known in the art or described herein, such as CRISPR, talens, or homologous recombination, may be employed.

Techniques for generating hypoimmune ABO blood group O Rh Factor (−) cells are described in Provisional App. No. 62/846,399 which is incorporated by reference herein in its entirety.

### D. Embodiments of the Invention

The SIRPα switch receptor cells, or derivatives thereof, of the invention may be used to treat, for example, tumors. Thus, provided herein are SIRPα switch receptor CAR-T or CAR-NK cells. In other aspects, the invention provides iPSC, ESC, HIP, iPSCO, ESCO, HIPO, iPSCO−, ESCO−, and HIPO− SIRPα switch receptor cells, or derivatives or differentiated cells thereof. They exhibit pluripotency but do not result in a host innate immune response when transplanted into an allogeneic host such as a human patient.

In one aspect, the present invention provides a SIRPα switch receptor cell, or derivative thereof, comprising a nucleic acid encoding a chimeric antigen receptor (CAR), wherein endogenous 3-2 microglobulin (β2M) gene activity and endogenous class II transactivator (CIITA) gene activity have been eliminated and a SIRPα engager molecule is provided on the cell surface. The CAR can comprise an extracellular domain, a transmembrane domain, and an intracellular signaling domain. In some embodiments, the extracellular domain binds to an antigen selected from the group consisting of CD19, CD20, CD22, CD38, CD123, CS1, CD171, BCMA, MUC16, ROR1, and WT1. In certain embodiments, the extracellular domain comprises a single chain variable fragment (scFv). In some embodiments, the transmembrane domain comprises CD3ζ, CD4, CD8α, CD28, 4-1BB, OX40, ICOS, CTLA-4, PD-1, LAG-3, and BTLA. In certain embodiments, the intracellular signaling domain comprises CD3ζ, CD28, 4-1BB, OX40, ICOS, CTLA-4, PD-1, LAG-3, and BTLA.

In certain embodiments, the CAR comprises an anti-CD19 scFv domain, a CD28 transmembrane domain, and a CD3 zeta signaling intracellular domain. In some embodiments, the CAR comprises an anti-CD19 scFv domain, a CD28 transmembrane domain, a 4-1BB signaling intracellular domain, and a CD3 zeta signaling intracellular domain.

In another aspect of the invention, provided is an isolated SIRPα switch receptor NK cell, CAR-NK cell, CAR-T cell, or hypoimmune versions thereof produced by in vitro differentiation of any one of the pluripotent cells described herein. In some embodiments, the SIRPα switch receptor cell is a cytotoxic HIPO− NK or CAR-T cell.

In various embodiments, the in vitro differentiation comprises culturing the SIRPα switch receptor cell, or derivative thereof, carrying a CAR construct in a culture media comprising one or more growth factors or cytokines selected from the group consisting of bFGF, EPO, Flt3L, IGF, IL-3, IL-6, IL-15, GM-CSF, SCF, and VEGF. In some embodiments, the culture media further comprises one or more growth factors or cytokines selected from the group consisting of a BMP activator, a GSK3 inhibitor, a ROCK inhibitor, a TGFβ receptor/ALK inhibitor, and a NOTCH activator.

In particular embodiments, the isolated SIRPα switch receptor CAR-T or CAR-NK cells are produced by in vitro differentiation of any one of iPSC, ESC, HIP, iPSCO, ESCO, HIPO, iPSCO−, ESCO−, or HIPO− SIRPα engager cells carrying the CAR-T constructs. In other embodiments, they are used to treat cancer.

In another aspect of the invention, provided is a method of treating a patient with cancer by administering a composition comprising a therapeutically effective amount of any of the isolated SIRPα switch receptor CAR-T or CAR-NK cells described herein. In some embodiments, the composition further comprises a therapeutically effective carrier.

In some embodiments, the administration step comprises intravenous administration, subcutaneous administration, intranodal administration, intratumoral administration, intrathecal administration, intrapleural administration, and intraperitoneal administration. In certain instances, the administration further comprises a bolus or by continuous perfusion.

In some embodiments, the cancer is a blood cancer selected from the group consisting of leukemia, lymphoma, and myeloma. In various embodiments, the cancer is a solid tumor cancer or a liquid tumor cancer.

In another aspect, the present invention provides a method of making any one of the isolated SIRPα switch receptor CAR-T or CAR-NK cells described herein. The method includes in vitro differentiating any one of the iPSC, ESC, HIP, iPSCO, ESCO, HIPO, iPSCO−, ESCO−, or HIPO− SIRPα engager cells of the invention. In vitro differentiation may comprise culturing the cells in a culture media comprising one or more growth factors or cytokines selected from the group consisting of bFGF, EPO, Flt3L, IGF, IL-2, IL-3, IL-6, IL-7, IL-15, GM-CSF, SCF, and VEGF. In some embodiments, the culture media further comprises one or more growth factors or cytokines selected from the group consisting of a BMP activator, a GSK3 inhibitor, a ROCK inhibitor, a TGFβ receptor/ALK inhibitor, and a NOTCH activator.

In some embodiments, the in vitro differentiating comprises culturing the iPSC, ESC, HIP, iPSCO, ESCO, HIPO, iPSCO−, ESCO−, or HIPO− SIRPα switch receptor cells on feeder cells. In various embodiments, the in vitro differentiating comprises culturing in simulated microgravity. In certain instances, the culturing in simulated microgravity is for at least 72 hours.

### E. Transplantation of HI SIRPα Switch Receptor Cells

As will be appreciated by those in the art that the HI SIRPα switch receptor cells cells are transplanted using techniques known in the art. In general, the HI SIRPα switch receptor cells of the invention are transplanted either intravenously or by injection at particular locations in the patient. When transplanted at particular locations, the cells may be suspended in a gel matrix to prevent dispersion while they take hold.

In order that the invention described herein may be more fully understood, the following examples are set forth. It should be understood that these examples are for illustrative purposes only and are not to be construed as limiting this invention in any manner.

### VIII. EXAMPLES

### Example 1: Generation of SIRPα Switch Receptor Cells

Two switch receptor proteins were designed to include the SIRPα signal peptide (SEQ ID NO:13) and the SIRPα ECD (SEQ ID NO:2). This sequence was followed by the TMDs and ICDs of CD16 (see SEQ ID NOS:11 and 3) or CD28 (see SEQ ID NOS:12 and 4).

The coding sequences for the SIRPα-CD16 or SIRPα-CD28 Switch Receptor proteins were cloned into lentiviral vectors that also included a red fluorescent protein (RFP) tag. Human NK cells were cultured in RPMI-1640 (Gibco, Cat No 11-875-101) with 10% FBS and 1% Pen/Strep and 0.1 μg/ml IL-2. NK cells were incubated with lentiviral particles at a multiplicity of infection of 20. The next day, polybrene (8 μg/ml, Millipore) was added to the media and the plate was centrifuged at 800 g for 30 min prior to the overnight incubation. NK cell populations expressing the SIRPα Switch Receptor were sorted on FACSAria (BD Biosciences) using the RFP tag.

### Example 2: SIRPα-CD16 AND SIRPα-CD28 Switch Receptor Cells Showed Enhanced Cell Killing Against CD47-Expressing Target Cells

Bioluminescence. K562 cancer cells were made to express firefly luciferase. 1×10⁵ K562 were plated in one 6-well plate and incubated overnight at 37° C. with 5% CO₂. The next day, the medium was changed and one vial of Fluc lentiviral particles expressing luciferase II gene under re-engineered EF 1a promotor (Gen Target, San Diego, CA) was added to 1.5 ml medium. After 36 hours, 1 ml of cell medium was added. After 24 hours, a complete medium change was performed. After 2 days, luciferase expression was confirmed by adding D-luciferin (Promega, Madison, WI). Signals were quantified in p/s/cm2/sr.

Some K562 were then also transduced to overexpress CD47 with lentiviral particles with a vector with the CD47 cDNA at a multiplicity of infection of 4 to achieve high CD47 expression (K562-CD47OV). K562 or K562-CD470V were used as targets in BLI killing assays.

Target cell survival was measured by luciferase bioluminescence imaging (BLI) and the killing was quantified by a drop in BLI signal. K562 killing assays were performed in 24 well plates using 5×10⁴ K562 cells or 5×10⁴ K562-CD470V as target cells, cocultured with 5×10⁴ primary NK-cells or 5×10⁴ switch receptor NK cells as effector cells (ratio 1:1, StemCell Technologies, Vancouver, BC, Canada) in RPMI 1640, 10% FCS hi, 1% Pen/Strep, 1% MEMNEAA (all Gibco), 1% Natrium Pyruvat and 0.2% 2-Mercaptoethanol (both Millipore). NK cells were pre-stimulated with human IL-2 overnight and during the assay (100 ng/mL, Peprotech). Plates were centrifuged down for 2 min at 1200 rpm. After 2h incubation at 37° C., 5 mg/mL D-Luciferin (Biosynth AG) were added to the wells and BLI signals were quantified with Ami HT (Spectral Instruments Imaging) in maximum photons s⁻¹ cm⁻² per steradian per 24-well. Data were normalized against wells with target cells only.

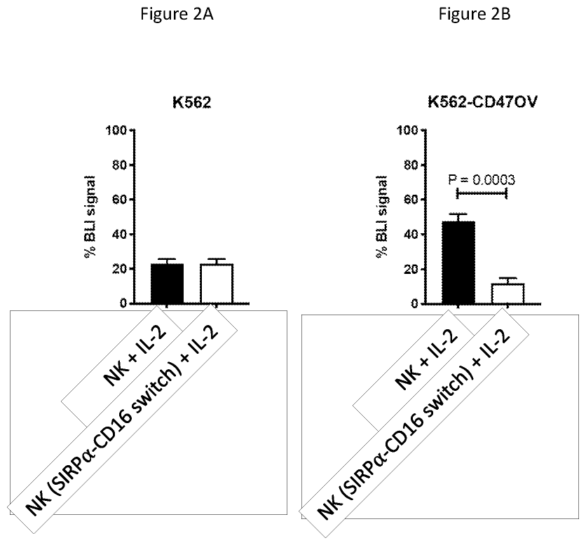

The in vitro killing capacity of primary human NK cells and primary human NK cells engineered to express the SIRPα-CD16 switch receptor was assessed. When K562 target cells were used as target cells in the presence of IL-2, both NKs and NK (SIRPα-CD16 switch) showed similar killing efficacy (FIG. 2A). K562 target cells overexpressing CD47 (K562-CD470V), however, were killed far more aggressively by NK (SIRPα-CD16 switch) cells (FIG. 2B). Thus, for target cells overexpressing CD47, the NK (SIRPα-CD16 switch) cells were more cytotoxic due to the CD16 intracellular signalling.

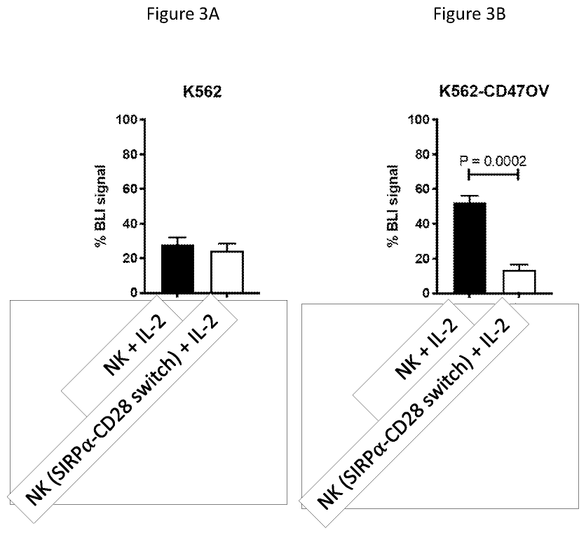

The in vitro killing capacity of primary human NK cells and primary human NK cells engineered to express the SIRPα-CD28 switch receptor was assessed. When K562 target cells were used as target cells in the presence of IL-2, both NKs and NK (SIRPα-CD28 switch) showed similar killing efficacy (FIG. 3A). K562 target cells overexpressing CD47 (K562-CD470V), however, were killed far more aggressively by NK (SIRPα-CD28 switch) cells (FIG. 3B). Thus, for target cells overexpressing CD47, the NK (SIRPα-CD28 switch) cells were more cytotoxic due to the CD28 intracellular signalling.

In vitro impedance assays. K562 or K562-CD470V killing was assayed using the XCelligence platform for in vitro impedance assays (ACEA BioSciences, San Diego, CA). The cells were attached to plastic dishes and formed confluent layers. Then, NK cells were added to the dishes in different effector cell-to-target cell ratios. Target cell killing was assessed by measuring the impedance between electrodes on special 96-well E-plates (ACEA BioSciences). Any drop in the cell index curve shows target cell cytotoxicity.

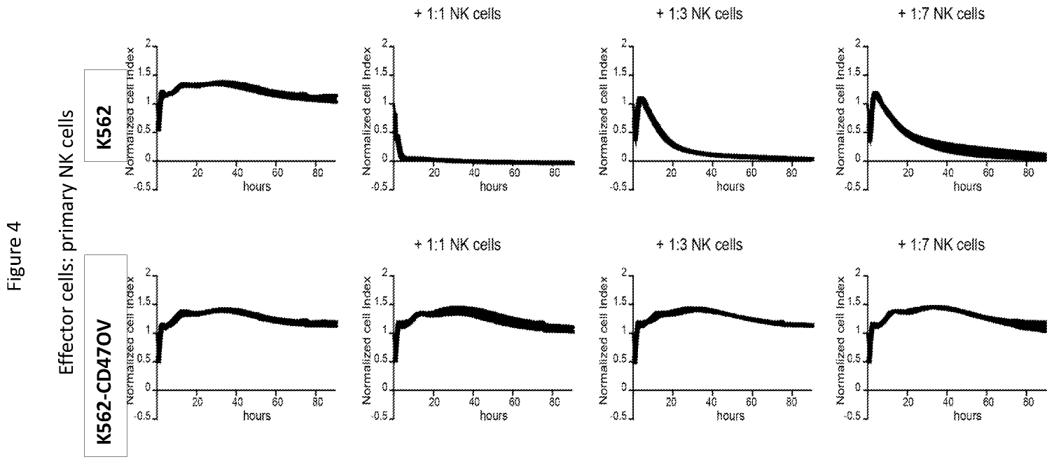

FIG. 4 shows stable survival of K562 and K562-OV if cultured without NK cells. When primary human peripheral blood NK cells were added in different effector cell-to-target cell ratios, the K562 were killed. Cell killing speed correlated with higher NK cell ratios. K562-CD47OV, however, was resistant and survived even in the 1:1 ratio.

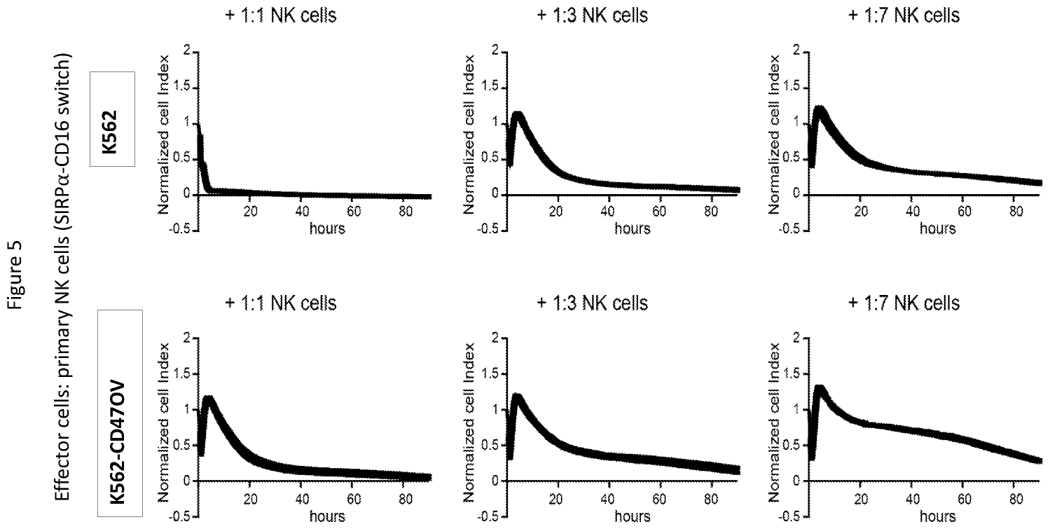

FIG. 5 shows the killing efficacy of human NK cells that express the SIRPα-CD16 switch receptor. Both K562 and K562-CD470V were killed effectively. CD47 overexpression in K562-CD470V did not protect these target cells from killing.

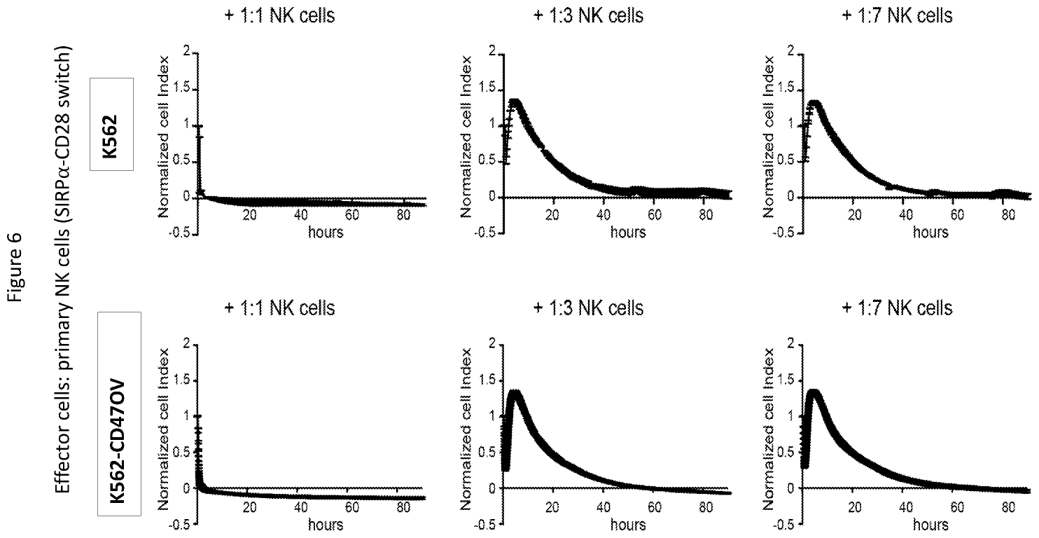

FIG. 6 shows the killing efficacy of human NK cells that express the SIRPα-CD28 switch receptor. Both K562 and K562-CD470V were killed effectively. CD47 overexpression in K562-CD470V did not protect these target cells from killing.

### Exemplary Sequences

SEQ ID NO: 1-Human SIRPα
>NP_001317657.1 tyrosine-protein phosphatase non-receptor type substrate 1
isoform 2 precursor [Homo sapiens]
MEPAGPAPGRLGPLLCLLLAASCAWSGVAGEEELQVIQPDKSVLVAAGETATLRCTATSLIPVG
PIQWFRGAGPGRELIYNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSPRDITLKWFKNGNELS
DFQTNVDPVGESVSYSIHSTAKVVLTREDVHSQVICEVAHVTLQGDPLRGTANLSETIRVPPTL
EVTQQPVRAENQVNVTCQVRKFYPQRLQLTWLENGNVSRTETASTVTENKDGTYNWMSWLLVNV
SAHRDDVKLTCQVEHDGQPAVSKSHDLKVSAHPKEQGSNTAAENTGSNERNIYIVVGVVCTLLV
ALLMAALYLVRIRQKKAQGSTSSTRLHEPEKNAREITQVQSLDTNDITYADLNLPKGKKPAPQA
AEPNNHTEYASIQTSPQPASEDTLTYADLDMVHLNRTPKQPAPKPEPSFSEYASVQVPRK
SEQ ID NO: 2-Human SIRPα Extracellular Domain
EEELQVIQPDKSVLVAAGETATLRCTATSLIPVGPIQWFRGAGPGRELIYNQKEGHFPRV
TTVSDLTKRNNMDFSIRIGNITPADAGTYYCVKFRKGSPDDVEFKSGAGTELSVRAKPS
APVVSGPAARATPQHTVSFTCESHGFSPRDITLKWFKNGNELSDFQTNVDPVGESVSYSI
HSTAKVVLTREDVHSQVICEVAHVTLQGDPLRGTANLSETIRVPPTLEVTQQPVRAENQ
VNVTCQVRKFYPQRLQLTWLENGNVSRTETASTVTENKDGTYNWMSWLLVNVSAHR
DDVKLTCQVEHDGQPAVSKSHDLKVSAHPKEQGSNTAAENTGSNERNIY
SEQ ID NO: 3-Human CD16 Intracellular Domain
KTNIRSSTRDWKDHKFKWRKDPQDK
SEQ ID NO: 4-Human CD28 Intracellular Domain
RSKRSRLLHSDYMNMTPRRPGPTRKHYQPYAPPRDFAAYRS
SEQ ID NO: 5-Human CD47
>NP_001768.1 leukocyte surface antigen CD47 isoform 1 precursor [Homo sapiens]
MWPLVAALLLGSACCGSAQLLENKTKSVEFTFCNDTVVIPCFVTNMEAQNTTEVYVKWKFKGRD
IYTFDGALNKSTVPTDESSAKIEVSQLLKGDASLKMDKSDAVSHTGNYTCEVTELTREGETIIE
LKYRVVSWFSPNENILIVIFPIFAILLFWGQFGIKTLKYRSGGMDEKTIALLVAGLVITVIVIV
GAILFVPGEYSLKNATGLGLIVTSTGILILLHYYVESTAIGLTSFVIAILVIQVIAYILAVVGL
SLCIAACIPMHGPLLISGLSILALAQLLGLVYMKFVASNQKTIQPPRKAVEEPLNAFKESKGMM
NDE
SEQ ID NO: 6 CD47 Extracellular Domain (ECD)
QLLFNKTKSVEFTFCNDTVVIPCFVTNMEAQNTTEVYVKWKFKGRDIYTFDGALNKSTVPTDFS
SAKIEVSQLLKGDASLKMDKSDAVSHTGNYTCEVTELTREGETIIELKYRVVSWFSPNE
SEQ ID NO: 7: CD47 Immunoglobulin Superfamily Domain
QLLFNKTKSVEFTFCNDTVVIPCFVTNMEAQNTTEVYVKWKFKGRDIYTFDGALNKSTVPTDFS
SAKIEVSQLLKGDASLKMDKSDAVSHTGNYTCEVTELTREGETIIELKYRVV
SEQ ID NO: 8: SIRPα-CD16 Switch Receptor (SIRPα (signal peptide + ECD)-CD16
(TMD + ICD))
MEPAGPAPGRLGPLLCLLLAASCAWSGVAGEEELQVIQPDKSVLVAAGETATLRCTATSLIPVG
PIQWFRGAGPGRELIYNQKEGHFPRVTTVSDLTKRNNMDFSIRIGNITPADAGTYYCVKERKGS
PDDVEFKSGAGTELSVRAKPSAPVVSGPAARATPQHTVSFTCESHGESPRDITLKWFKNGNELS
DFQTNVDPVGESVSYSIHSTAKVVLTREDVHSQVICEVAHVTLQGDPLRGTANLSETIRVPPTL
EVTQQPVRAENQVNVTCQVRKFYPQRLQLTWLENGNVSRTETASTVTENKDGTYNWMSWLLVNV
SAHRDDVKLTCQVEHDGQPAVSKSHDLKVSAHPKEQGSNTAAENTGSNERNIYYQVSFCLVMVL
LFAVDTGLYFSVKTNIRSSTRDWKDHKFKWRKDPQDK
SEQ ID NO: 9: SIRPα-CD28 Switch Receptor (SIRPα (signal peptide + ECD)-CD28
(TMD + ICD))
MEPAGPAPGRLGPLLCLLLAASCAWSGVAGEEELQVIQPDKSVLVAAGETATLRCTATSLIPVG
PIQWFRGAGPGRELIYNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSPRDITLKWFKNGNELS
DFQTNVDPVGESVSYSIHSTAKVVLTREDVHSQVICEVAHVTLQGDPLRGTANLSETIRVPPTL
EVTQQPVRAENQVNVTCQVRKFYPQRLOLTWLENGNVSRTETASTVTENKDGTYNWMSWLLVNV
SAHRDDVKLTCQVEHDGQPAVSKSHDLKVSAHPKEQGSNTAAENTGSNERNIYFWVLVVVGGVL
ACYSLLVTVAFIIFWVRSKRSRLLHSDYMNMTPRRPGPTRKHYQPYAPPRDFAAYRS
SEQ ID NO: 10: CD47 Transmembrane Domain (TMD)
NILIVIFPIFAILLFWGQFGIKTLKYRSGGMDEKTIALLVAGLVITVIVIVGAILFVPGEYSLK
NATGLGLIVISTGILILLHYYVFSTAIGLTSFVIAILVIQVIAYILAVVGLSLCIAACIPMHGP
LLISGLSILALAQLLGLVYM
SEQ ID NO: 11: CD16 Transmembrane Domain (TMD)
VSFCLVMVLLFAVDTGLYFSV
SEQ ID NO: 12: CD28 Transmembrane Domain (TMD)
FWVLVVVGGVLACYSLLVTVAFIIFWV
SEQ ID NO: 13 SIRPα Signal Peptide
MEPAGPAPGRLGPLLCLLLAASCAWSGVAG
SEQ ID NO: 14: IL-2 Signal Peptide
MYRMQLLSCIALSLALVINS

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A recombinant CD47 engager receptor protein, comprising an extracellular domain (ECD) that binds to a CD47 protein, a transmembrane domain (TMD), and an intracellular domain (ICD) that enhances an immune response within an immune cell when the ECD recognizes said CD47 protein on a target cell.

2. The recombinant CD47 engager receptor protein of claim 1, wherein said CD47 protein comprises at least a 90% sequence identity to SEQ ID NO:6

3. The recombinant CD47 engager receptor protein of claim 2, wherein said CD47 sequence comprises the sequence of SEQ ID NO:6.

4. The recombinant CD47 engager receptor protein of any one of claims 1-3, wherein said CD47 protein is a recombinant protein.

5. The recombinant CD47 engager receptor protein of any one of claims 1-4, wherein said ECD has at least a 90% sequence identity to SEQ ID NO:2.

6. The recombinant CD47 engager receptor protein of claim 5, wherein said ECD comprises the sequence of SEQ ID NO:2.

7. The recombinant CD47 engager receptor protein of any one of claims 1-6, wherein said ICD comprises at least a 90% sequence identity to SEQ ID NO:3.

8. The recombinant CD47 engager receptor protein of claim 7, wherein said ICD comprises the sequence of SEQ ID NO:3.

9. The recombinant CD47 engager receptor protein of any one of claims 1-6, wherein said ICD comprises at least a 90% sequence identity to SEQ ID NO:4.

10. The recombinant CD47 engager receptor protein of claim 9, wherein said ICD comprises the sequence of SEQ ID NO:4.

11. The recombinant CD47 engager receptor protein of any one of claims 1-10, wherein said TMD comprises at least a 90% sequence identity to SEQ ID NOS:11 or 12.

12. The recombinant CD47 engager receptor protein of claim 11, wherein said TMD comprises the sequence of SEQ ID NOS:11 or 12.

13. The recombinant CD47 engager receptor protein of any one of claims 1-12, further comprising a signal peptide.

14. The recombinant CD47 engager receptor protein of claim 13, wherein said signal peptide comprises at least a 90% sequence identity to SEQ ID NOS:13 or 14.

15. The recombinant CD47 engager receptor protein of claim 14, wherein said signal peptide comprises the sequence of SEQ ID NOS:13 or 14.

16. A cell, comprising the recombinant CD47 engager receptor protein of any one of claims 1-15.

17. The cell of claim 16, wherein said cell is an immune cell.

18. The cell of claim 17, wherein said cell is a natural killer (NK) cell, a macrophage, or a T cell.

19. The cell of claim 17, wherein said cell is an NK cell.

20. The cell of claim 19, wherein said NK cell comprises a chimeric antigen receptor (CAR-NK cell).

21. The cell of claim 17, wherein said cell is a T cell.

22. The cell of claim 21, wherein said T cell comprises a chimeric antigen receptor (CAR-T cell).

23. The cell of any one of claims 16-22, further comprising a reduced or eliminated HLA-I or HLA-II expression.

24. The cell of any one of claims 16-23, wherein said cell is ABO blood group type O.

25. The cell of any one of claims 16-24, wherein said cell is Rhesus factor negative (Rh−).

26. The cell of any one of claims 16-25, wherein said cell has a reduced or eliminated ABO blood group antigen selected from the group consisting of A1, A2, and B.

27. The cell of any one of claims 16-26, wherein said cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

28. The cell of any one of claims 16-27, wherein the cell is a hypoimmunogenic (HI) cell comprising: an endogenous Major Histocompatibility Complex Class I (HLA-I) function that is reduced when compared to an unmodified parental cell and an endogenous Major Histocompatibility Complex Class II (HLA-II) function that is reduced when compared to said unmodified parental cell.

29. The cell of any one of claims 16-28, wherein said cell comprising said CD47 engager receptor protein further comprises modulated expression of one or more of HLA-I human leukocyte antigens, HLA-II human leukocyte antigens, CD64, CD47, CD38, CCR5, CXCR4, NLRC5, CIITA, β2M, HLA-A, HLA-B, HLA-C, HLA-E, HLA-G, PD-L1, CTLA-4-Ig, CD47, CI-inhibitor, IL-35, RFX-5, RFXAP, RFXANK, NFY-A, NFY-B, NFY-C, IRF-1, OX40, GITR, 4-1BB, CD28, B7-1, B7-2, ICOS, CD27, HVEM, SLAM, CD226, PD1, CTL4, LAG3, TIGIT, TIM3, CD160, BTLA, CD244, CD30, TLT, VISTA, B7-H3, PD-L2, LFA-1, CD2, CD58, ICAM-3, TCRA, TCRB, FOXP3, HELIOS, ST2, PCSK9, APOC3, CD200, FASLG, CLC21, MFGE8, SERPIN B9, TGFβ, CD73, CD39, LAG3, IL1R2, ACKR2, TNFRSF22, TNFRSF23, TNFRS10, DAD1, and/or IFNγR1 d39 relative to a wild-type stem cell, wherein said engager cell is ABO blood group type O or Rhesus factor negative (Rh−).

30. The cell of any one of claims 16-29, further comprising an elevated expression of an antibody Fc receptor on the cell surface, wherein said Fc receptor helps to evade antibody dependent cellular cytotoxicity (ADCC) or complement mediated cytotoxicity (CDC).

31. The cell of claim 30, wherein said Fc receptor is CD16, CD32, or CD64.

32. The cell of claim 16, wherein said cell is pluripotent.

33. The cell of claim 32, wherein said cell is a hypoimmune pluripotent (HIP) cell.

34. The cell of claim 33, wherein said cell is a hypoimmune pluripotent cell having an ABO blood type O (HIPO).

35. The cell of claim 33, wherein said cell is a hypoimmune pluripotent cell is Rh factor negative (HIP−).

36. The cell of any one of claims 32 to 35, wherein said cell is a hypoimmune pluripotent cell having an ABO blood type O and is Rh factor negative (HIPO−).

37. The cell of claim 16, wherein said cell is a pluripotent (PSC) cell, induced PSC (iPSC), or an embryonic stem cell (ESC).

38. The cell of any one of claims 16-31, wherein said cell is differentiated from a pluripotent cell.

39. A pharmaceutical composition, comprising the cell of any one of claims 16-38 and a pharmaceutically-acceptable carrier.

40. A medicament, comprising the SIRP-α switch receptor cell of any one of claims 16-38 and a pharmaceutically-acceptable carrier.

41. A method of treating a disease in a subject, comprising transplanting the cell of any one of claims 16-38 into said subject.

42. The method of claim 41, wherein said disease is cancer.

43. The method of claim 42, wherein said cancer is leukemia, lymphoma, myeloma, a solid tumor, or a liquid tumor.

44. A use of the cells of any one of claims 16-38 for preparing a pharmaceutical composition for treating a disease in a subject.

45. The use of claim 44, wherein said disease is cancer.

46. A use of the cell of any one of claims 16-38 for treating a disease in a subject.

47. The use of claim 46, wherein said disease is cancer.

48. The use of either one of claims 45 or 47, wherein said cancer is leukemia, lymphoma, myeloma, a solid tumor, or a liquid tumor.


SEQUENCE LISTING (14 sequences)

SEQ ID NO: 1 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 343 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 3 - protein, 25 aa, Homo sapiens
KTNIRSSTRDWKDHKFKWRKDPQDK

SEQ ID NO: 4 - protein, 41 aa, Homo sapiens
RSKRSRLLHSDYMNMTPRRPGPTRKHYQPYAPPRDFAAYRS

SEQ ID NO: 5 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 6 - protein, 123 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

SEQ ID NO: 7 - protein, 116 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVV

SEQ ID NO: 8 - protein, 421 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYYQVSFCLVMVLLFAVDTGLYFSVKTNIRSSTRDWKDHKFKWRKDPQDK

SEQ ID NO: 9 - protein, 441 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYFWVLVVVGGVLACYSLLVTVAFIIFWVRSKRSRLLHSDYMNMTPRRPGPTRKHYQPYAPPRDFAAYRS

SEQ ID NO: 10 - protein, 148 aa, Homo sapiens
NILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYM

SEQ ID NO: 11 - protein, 21 aa, Homo sapiens
VSFCLVMVLLFAVDTGLYFSV

SEQ ID NO: 12 - protein, 27 aa, Homo sapiens
FWVLVVVGGVLACYSLLVTVAFIIFWV

SEQ ID NO: 13 - protein, 30 aa, Homo sapiens
MEPAGPAPGRLGPLLCLLLAASCAWSGVAG

SEQ ID NO: 14 - protein, 20 aa, Homo sapiens
MYRMQLLSCIALSLALVTNS